(19) United States
(12) Patent Application Publication
(10) Pub. No.: US 2017/0333239 A1
(43) Pub. Date: Nov. 23, 2017

(54) A DEVICE FOR TREATING OBESITY AND A CORRESPONDING DELIVERY SYSTEM

(71) Applicant: Federico CARDINALI, Torino (IT)

(72) Inventor: Federico CARDINALI, Torino (IT)

(21) Appl. No.: 15/532,794

(22) PCT Filed: Dec. 2, 2015
(86) PCT No.: PCT/IB2015/059282

§ 371 Date: Jun. 2, 2017

(30) Foreign Application Priority Data
Dec. 4, 2014 (IT) TO2014A001014

(51) Int. Cl. A61F 5/00

(52) U.S. Cl. CPC A61F 5/0033; A61F 5/0076; A61F 5/0089

(57) ABSTRACT

A device for the treatment of obesity, the device including an inflatable member positionable within the stomach of a patient and an elongated flexible sleeve positionable in the intestine of the patient and having a proximal end coupled to said inflatable member, a distal end and a side wall extending from said proximal end to said distal end and defining a lumen. The inflatable member includes an aperture for the passage of food, said aperture being in communication with the lumen of the elongated flexible sleeve.

### TECHNICAL FIELD

The present description relates to devices for treating obesity in humans.

One or more embodiments may relate to devices insertable in the stomach and the e.g. small intestine of a patient in order to reduce the patient's weight and to meliorate the metabolism of glucose, blood pressure and other overweight-related diseases.

### BACKGROUND

The incidence of obesity is dramatically increasing as a worldwide pandemic of the 21st century. More than 60 million people worldwide currently face the dual challenge of managing type 2 diabetes mellitus and other obesity related diseases. An increased incidence of some neoplasms has also been reported. Furthermore, life expectancy is reduced to up to 20 years in severely obese. Dietetic protocols or anti-obesity medications usually fail to induce sustained weight loss at long-term, this latter being obtained consistently only with bariatric surgery.

Bariatric operations include restrictive and malabsorptive procedures.

Restrictive operations are intended to limit food intake by modifying the gastric anatomy, reducing the stomach size and giving the patient a feeling of fullness after small meals.

Malabsorptive surgery causes weight loss because the surgically altered gastric, jejunal and ileal anatomy significantly impairs the absorption of nutrients.

The combination of both restrictive and malabsorptive surgery is currently the most frequently used approach for obesity treatment.

Although highly effective, bariatric surgery may be associated with significant severe complications such as embolism or thrombosis, leakage or abscess, wound infections, cardiac and pulmonary complications etc. Other late surgery complications are malabsorption, dumping syndrome, ulcers, infections and stenoses.

The insertion of specific devices in the stomach and intestine such as, for example, the device disclosed in US-A-2014/0276338 may allow to obtain weight loss and to meliorate other obesity related disorders. A drawback of these types of devices may lie in that they are anchored to the stomach and intestine wall and this approach may induce ulcers at the site of anchoring. In addition, these types of devices may move and migrate inside the stomach and the intestine and induce intestinal obstruction, severe injuries during removal, etc.

### OBJECT AND SUMMARY

In view of the foregoing, the need is felt for alternative devices which may be positioned in the stomach and the intestine in order to obtain e.g. weight loss, meliorated metabolism of glucose and improvements in obesity related disorders of a patient.

According to one or more embodiments, such an object may be achieved by means of a device having the features set forth in the claims that follow.

Ore or more embodiments may also relate to a corresponding delivery system.

The claims are an integral part of the disclosure of one or more embodiments as provided herein.

One or more embodiments, as disclosed herein may include an inflatable member positionable within the stomach of a patient and an elongated flexible sleeve positionable in the intestine of the patient and having a proximal end coupled to said inflatable member, a distal end and a side wall extending from said proximal end to said distal end and defining a lumen, wherein the inflatable member includes an aperture for the passage of food, said aperture being in communication with the lumen of the elongated flexible sleeve.

In one or more embodiments, a device as disclosed herein may restrict food and caloric intake in the stomach and reduce the absorption of the food components in the stomach and small intestine therefore combining restrictive and malabsorptive procedures for the treatment of obesity.

In one or more embodiments, the device may be deployed and positioned through a delivery system including:
a tube including a proximal aperture and a distal aperture, said tube containing the elongate flexible sleeve and the inflatable member in a uninflated configuration,
a pusher member insertable into said tube and slidable therein to urge the elongate flexible sleeve and said inflatable member along the tube in order to load said elongate flexible sleeve and said inflatable member in a uninflated configuration into an endoscope for subsequent placement in the intestine and the stomach.

In one or more embodiments, a device as disclosed herein may thus be easily inserted and positioned through the operative channel of an endoscope introduced through the mouth into the stomach and the first parts of the small intestine. Moreover, if necessary, the device may be modified and finally removed using minimally invasive techniques.

Consequently, the device such as the system for its deployment and positioning herein disclosed allow to obtain scarless, noninvasive, safe, repeatable and more cost-effective treatment of obesity with the further advantages to provide a decrease in morbidity, in the hospitalization duration and costs.

One or more embodiments may include an inflatable member positionable within the stomach of a patient and an elongated flexible sleeve positionable in the intestine of the patient.

In one or more embodiments, the elongated flexible sleeve may have a proximal end which is coupled to the inflatable member and also includes a distal end and a side wall extending from the proximal end to said distal end and defining a lumen.

In one or more embodiments, the inflatable member may include an aperture for the passage of food and said aperture is in communication with the lumen of the elongated flexible sleeve.

In one or more embodiments, the inflatable member may have an annular shape and may be realized in a single body or may include a plurality of stacked inflatable bodies.

In one or more embodiments, the inflatable member may have a funnel shape to adapt to the gastric body and the antrum and may be made of flexible, acid, alkaline and compression resisting material; these material are compatible with use in the human stomach as well as sufficient elastic stretch capability to expand.

In one or more embodiments, the inflatable member may include at least one fill port for injecting an inflating fluid into the inflatable member through a filling tube. When the patient begins to eat, the internal stomach volume available for food is reduced by the presence of the inflated inflatable member, thus discouraging the patient from overeating.

### BRIEF DESCRIPTION OF THE FIGURES

One or more embodiments will now be described, by way of example only, with reference to the enclosed Figures, wherein:

FIG. 1 is a side view of a device according to embodiments as disclosed herein shown inserted within the stomach and the intestine of a patient,

FIG. 2 is a side view of a device according to embodiments as disclosed herein showing the insufflation of an inflatable member,

FIG. 3 is a perspective illustration of a device according to embodiments as disclosed herein,

FIG. 4 is a sectional side view of a delivery system according to embodiments as disclosed herein.

### DETAILED DESCRIPTION OF EMBODIMENTS

In the following description, numerous specific details are given to provide a thorough understanding of examples of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

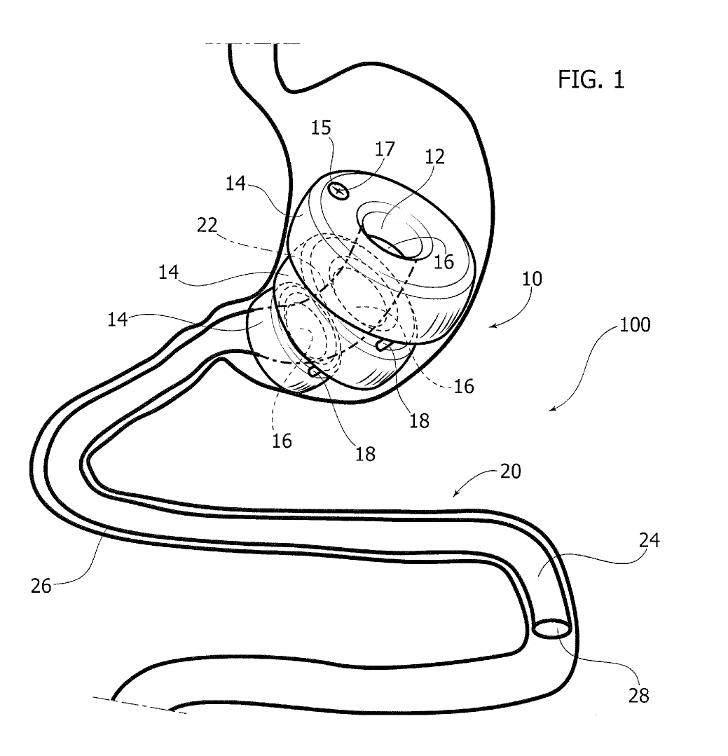

In FIGS. 1 to 3, the reference number 100 indicates as a whole a device for the treatment of obesity.

In one or more embodiments, the inflatable member 10 may include a single or a plurality (e.g. three) stacked inflatable bodies 14 having a substantially annular shape and central apertures 16 aligned to define the aperture 12 for the passage of food (FIGS. 1 to 3).

In one or more embodiments, the stacked inflatable bodies 14 may include/be made of any of human body compatible materials such as, for example, silicone and polyurethane.

In one or more embodiments, the stacked inflatable bodies 14 may be fabricated to have uniform equal diameters or alternatively as suggested in FIGS. 1 to 3 they may have serially decreasing diameters in order to provide a funnel-shaped pattern.

Thanks to such a funnel-shaped pattern the stacked inflatable bodies 14 may adapt to the anatomy of the stomach therefore allowing the maintenance of the proper positioning within the stomach.

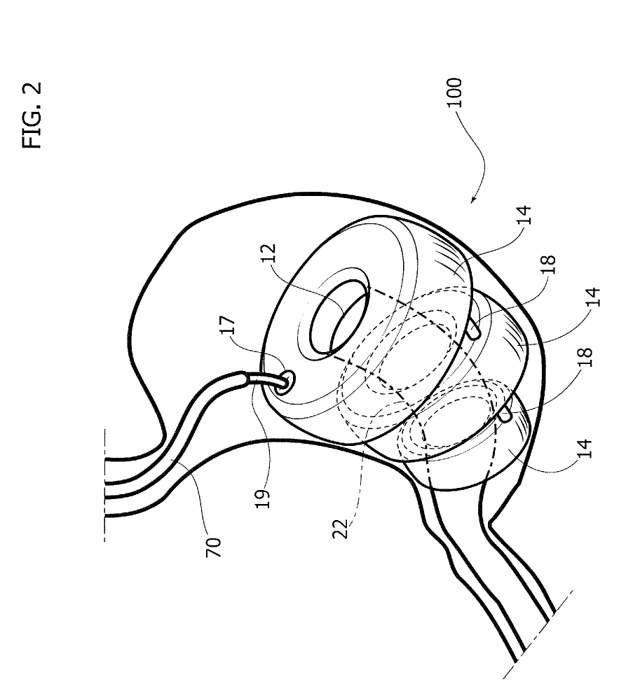

In one or more embodiments, the plurality of stacked inflatable bodies 14 may include at least one fill port 15. Through the fill port 15 an inflating fluid can be filled into the inflatable bodies 14 by means of a filling tube 19 (shown in FIG. 2). In one or more embodiments, the fill port 15 may have a closing valve 17 associated thereto.

In one or more embodiments, the plurality of stacked inflatable bodies 14 may be connected by flexible hollow connectors means 18. The inflating fluid may thus flow through the connectors means 18 and fill the inflatable bodies 14 allowing their expansion.

The inflating fluid may be selected among any of human body compatible liquids, for example physiological solution, or medical gases such as, for example, nitrogen.

In one or more embodiments, the elongated flexible sleeve 20 may have a proximal end 22 coupled to the inflatable member 10, a distal end 24 and a side wall 26 extending from the proximal end 22 to said distal 24 end and defining a lumen 28.

The elongated flexible sleeve 20 may induce malabsorption of consumed food by serving as a barrier between the food and the small intestine.

In one or more embodiments, the elongated flexible sleeve 20 may include a flexible, acid and alkaline resisting material.

In one or more embodiments, the material may be permeable, semipermeable (selectively permeable) or non-permeable. In one or more embodiments, permeability may be selected as a function of the desired absorption rate of the products passing through the elongate flexible sleeve 20.

In one or more embodiments, the elongate flexible sleeve 20 may be divided in sections where a section is permeable, a section is semipermeable and a section is non-permeable.

The elongated flexible sleeve 20 may be of any suitable width and length, optionally with a length of from about 30 cm to about 70 cm, preferably about 50 cm to about 60 cm.

In one or more embodiments, the proximal end 22 of the elongated flexible sleeve 20 may be arranged inside the passage defined by the central apertures 16 of the plurality of stacked inflatable bodies 14, as shown in FIGS. 1 to 3. The proximal end 22 of the elongated flexible sleeve 20 may therefore accommodate the passage of food through the stomach, with food advancing by physiologic peristalsis within the lumen 28 of the elongated flexible sleeve 20 along the aperture of the funnel-shaped inflatable member 10.

In one or more embodiments, the proximal end 22 of the elongated flexible sleeve 20 may be sealably attached to the stacked inflatable bodies 14.

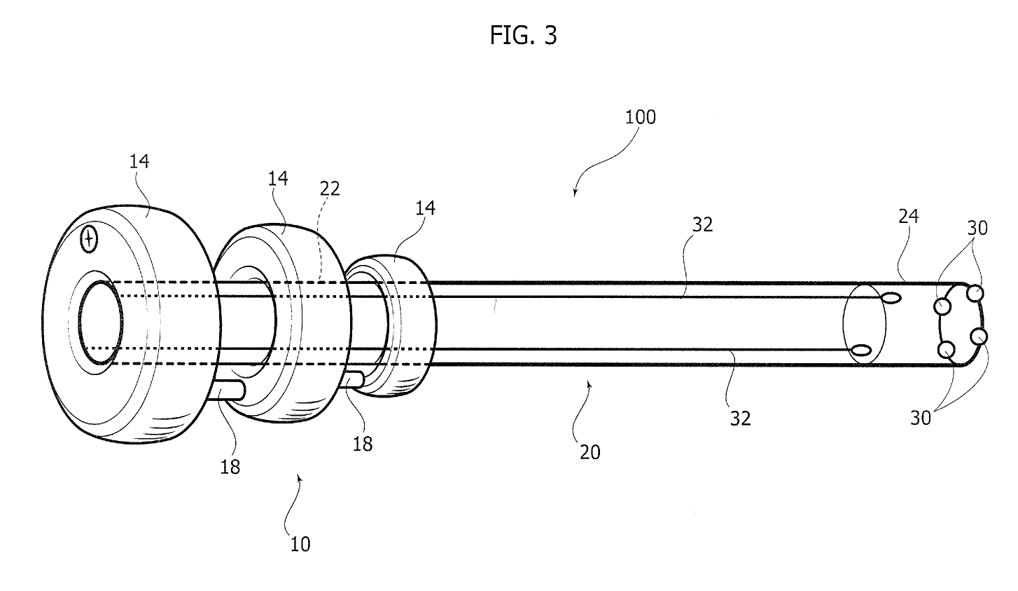

In one or more embodiments, the elongated flexible sleeve 20 may include at least one anchor weight 30 connected to the distal end 24 to counter the retraction of the elongated flexible sleeve 20 when positioned in the intestine (FIG. 3).

In one or more embodiments, a single anchor weight may be employed having an annular or any other suitable configuration, or a plurality of anchor weights 30 may be disposed in spaced relation around the perimeter margin of the distal end 24, as shown in FIG. 3.

The anchor weights 30 may be constructed of any suitable material such as a medical grade synthetic resin or metal.

In one or more embodiments, the elongated flexible sleeve 20 may include a shape-memory material/wires intended to facilitate positioning and to counter collapse and displacement of the device.

In one or more embodiments, the side wall 26 of the elongated flexible sleeve 20 may include at least one support e.g. shape-memory wire 32 able to counter twisting of the elongate flexible sleeve once positioned in the intestine.

In one or more embodiments, as shown in FIG. 3, support e.g. shape-memory wires 32 extend from the proximal end 22 to the proximity of the distal end 24 of the elongated flexible sleeve 20.

The distal part of the support wires 32 may be of a rounded/oval shape in order to counter the perforation of the side wall 26 of the elongated flexible sleeve 20 (FIG. 3).

The support wire 32 may be constructed of any material capable of being formed into a shape-retaining support, such as a synthetic resin or so-called “memory” resin, metal, metal alloy such as Nitinol, or any suitable material.

In one or more embodiments a device as disclosed herein may have an outer surface free from anchoring means both to the stomach and to the intestine wall; the presence of any such anchoring is in fact not mandatory since the configuration of the device herein disclosed allows the maintenance of the proper position once inserted in the stomach and the intestine of the patient.

In one or more embodiments, the inflatable member 10, the elongate flexible sleeve 20 and, preferably, the filling tube 19 for inflating the inflatable member 10 may be of different colors and are distinguishable during positioning.

A device 100 according to one or more embodiments as disclosed herein may be easily inserted, deployed and positioned into the patient stomach and intestine via the operative channel of an endoscope introduced through the mouth into the stomach and first parts of the small intestine.

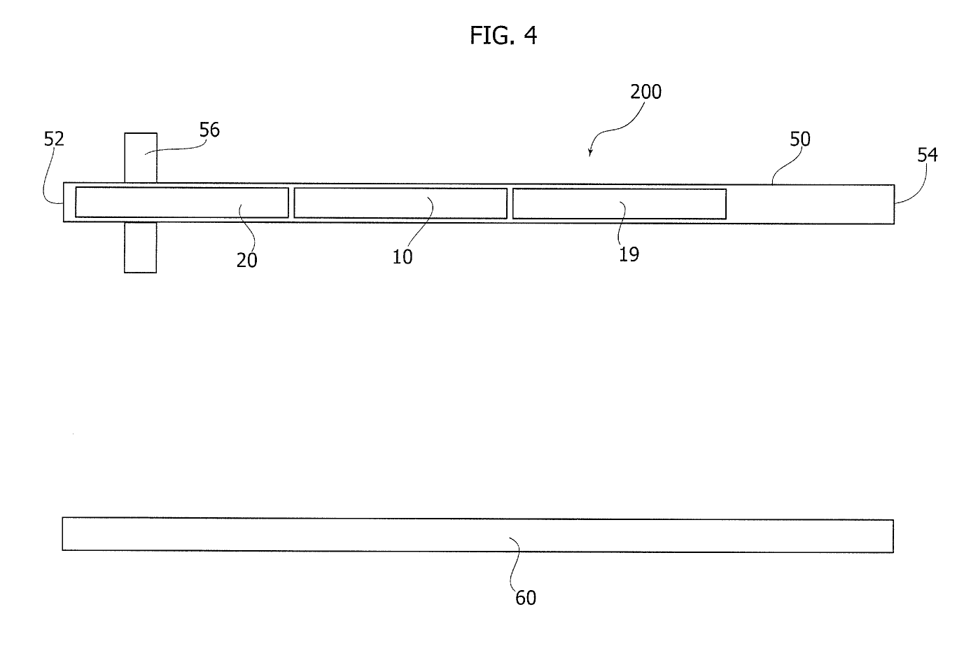

One or more embodiments of a delivery system 200 for a device 100 as disclosed herein may include a tube 50 having a proximal aperture 52 and a distal aperture 54 and containing the elongate flexible sleeve 20 and the inflatable member 10 in a uninflated configuration (FIG. 4).

One or more embodiments of such a delivery system 200 may include a pusher member 60 insertable into the tube 50 and slidable therein to urge the elongate flexible sleeve 20 and the inflatable member 10 along the tube 50 in order to load the elongate flexible sleeve 20 and the inflatable member 10 in a uninflated configuration into an endoscope (70, shown in FIG. 2) for subsequent placement in the intestine and the stomach.

Once the endoscope reaches the third duodenal portion or even deeper, the endoscopist connects the delivery system 200 to the proximal part of the operative channel of the endoscope.

In one or more embodiments, the proximal aperture 52 of the tube 50 may be sized and dimensioned in order to fit over the operative channel of the endoscope to provide a tight fit or a friction-fit engagement between the proximal aperture 52 of the tube 50 and the operative channel of the endoscope.

In one or more and embodiments, the tube 50 may include a security system, preferably a stop member (56), in order to avoid an undesirable insertion of the tube (50) into the operative channel of the endoscope.

The pusher member 60 may therefore be inserted into the tube 50 to urge the elongate flexible sleeve 20 and the inflatable member 10 along the tube 50 in order to load the elongate flexible sleeve 20 and the inflatable member 10 in a uninflated configuration into the endoscope.

In one or more embodiment, as exemplified in FIG. 4, the tube 50 may also contain the filling tube 19 so that the pusher member 60 also urges the filling tube 19 inside the endoscope. Once the elongate flexible sleeve 20, the inflatable member 10 and the filling tube from the tube 50 are entirely introduced inside the endoscope, the tube 50 can be removed.

The pusher member 60 pushes gently the device 100 inside the endoscope to force the device 100 to a specific placement in the stomach and small intestine.

An endoscopist may slowly retrieve back the endoscope and may thus be guided in the correct positioning of the device 100 by the different colors of the elongate flexible sleeve 20, the inflatable member 10 and the filling tube, with each color making it possible to identify the part of the device 100 which is being released. Once the inflatable member 10 is fully released into the stomach only the filling tube 19 will be seen going out from the endoscope.

Through the filling tube 19 an inflating fluid may be injected so that the inflatable member 10 is inflated under direct vision until the desired volume is achieved. The filling tube 19 may be similarly detached under direct endoscopic vision.

A delivery system 200 as exemplified herein allows simple insertion of the device 100 for the treatment of obesity with minimal discomfort for the patient, while also substantially avoiding health risk complications for the patient associated to other surgical insertion systems.

The device 100 of one or more embodiments may be removed by deflating the inflatable mean 10 e.g. by perforation with an endoscopic needle, so that the device 100 becomes adapted to be removed with a foreign body forceps or a snare or other accessories.

Various modifications and additions can be made to the exemplary embodiments discussed without departing from the extent of protection.

The extent of protection is defined by the annexed claims.

1. A device for the treatment of obesity, the device including:
an inflatable member positionable within the stomach of a patient, and
an elongated flexible sleeve positionable in the intestine of the patient and having a proximal end coupled to said inflatable member, a distal end and a side wall extending from said proximal end to said distal end and defining a lumen,
wherein the inflatable member includes an aperture for the passage of food, said aperture being in communication with the lumen of the elongated flexible sleeve.

2. The device of claim 1, wherein the inflatable member has an annular shape.

3. The device of claim 1, wherein the inflatable member includes a plurality of stacked inflatable bodies having a substantially annular shape, with central apertures aligned to define said aperture for the passage of food.

4. The device of claim 3, wherein the plurality of stacked inflatable bodies are connected by flexible hollow connector means.

5. The device of claim 1, wherein the inflatable member has a funnel shape to adapt to the gastric body and the antrum.

6. The device of claim 1, wherein the inflatable member includes at least one fill port for injecting an inflating fluid into the inflatable member through a filling tube, said fill port having preferably a closing valve associated therewith.

7. The device of claim 1, wherein the elongated flexible sleeve includes at least one anchor weight connected to the distal end to counter the retraction of the elongated flexible sleeve when positioned in the intestine.

8. The device of claim 1, wherein the side wall of the elongated flexible sleeve includes at least one support wire, preferably extending from the proximal end to the proximity of the distal end, to counter twisting of the elongated flexible sleeve once positioned in the intestine.

9. The device of claim 1, having an outer surface free of anchoring means to the stomach and to the intestine wall.

10. The device of claim 1, wherein said inflatable member, said elongate flexible sleeve and, preferably, said filling tube for inflating said inflatable member are of different colors and distinguishable during positioning.

11. A delivery system of a device according to claim 1, the system including:
a tube including a proximal aperture, a distal aperture, said tube containing the elongate flexible sleeve and said inflatable member in a uninflated configuration,
a pusher member insertable into said tube and slidable therein to urge the elongate flexible sleeve and said inflatable member along the tube in order to load said elongate flexible sleeve and said inflatable member in a uninflated configuration into an endoscope for subsequent placement in the intestine and the stomach.

12. The delivery system of claim 11, wherein the tube further contains the filling tube and wherein sliding of said pusher member also urges the filling tube in order to load the latter into the endoscope.

13. The delivery system of claim 11, wherein the tube includes a security system, preferably a stop member, in order to avoid an undesirable insertion of the tube into the endoscope.
